(19) United States
(12) Patent Application Publication
(10) Pub. No.: US 2024/0047082 A1
(43) Pub. Date: Feb. 8, 2024

(54) METHOD AND DEVICE FOR CLEANING DRUG-TARGET INTERACTION DATA

(71) Applicant: Ainnocence Technologies LLC, MIAMI, FL (US)

(72) Inventors: Yang Jiao, Shanghai (CN); Lurong Pan, Vestavia Hill, AL (US)

(21) Appl. No.: 18/164,689

(22) Filed: Feb. 6, 2023

(30) Foreign Application Priority Data
Aug. 3, 2022 (CN) 2022109295703

(51) Int. Cl. G16H 70/40; G16H 20/10

(52) U.S. Cl. CPC G16H 70/40; G16H 20/10

(57) ABSTRACT

The present invention provides a cleaning method for drug-target interaction data, which comprises the following steps: provide an original collection of drug-target interaction data; screen and filter the original drug-target interaction data set according to a predetermined cleaning rule to obtain a drug-target interaction data set to be studied; wherein, the predetermined cleaning rule is based on the data structure of the adjacency matrix of the graph. The present invention further provides a cleaning system for drug-target interaction data. The present invention provides a method and system for properly describing and comparing the structure, function, etc. of drug target proteins, so as to the differences in target proteins can be quantified.

### CROSS REFERENCE TO RELATED APPLICATIONS

This Application claims priority from a patent application filed in China having Patent Application No. 2022109295703 filed on Aug. 3, 2022 and titled “METHOD AND DEVICE FOR CLEANING DRUG-TARGET INTERACTION DATA”.

### TECHNICAL FIELD OF THE INVENTION

The invention belongs to the field of artificial intelligence-assisted drug research and development, and in particular relates to a drug molecule design driven by data and based on machine learning and artificial intelligence, evaluation and research of drug-target interaction structure-activity relationship, construction and collation of large drug data sets, and the like.

### BACKGROUND OF THE INVENTION

At present, there are a large number of published or private drug-target interaction data in the field, but attempts to directly mix these data for modeling and training, and to predict the efficacy of new potential drugs are often frustrated by the differences in the chemical space between target proteins and corresponding drug molecules. Different types of targets may have different or even completely opposite mechanisms in the structure-activity relationship, which leads to problems such as over-fitting or insufficient generalization ability of the model and poor performance of the model for predicting new targets.

Based on the above, the present application provides a technical solution to solve the above technical problems.

### SUMMARY OF THE INVENTION

The first objective of the present invention is to obtain a cleaning method for drug-target interaction data, which can properly describe and compare the structure and function of drug target proteins so that differences in target proteins can be quantified.

The second object of the present invention is to obtain a cleaning system for drug-target interaction data, which can properly describe and compare the structure, function, etc. of drug target proteins so that differences in target proteins can be quantified.

The first aspect of the present invention provides a cleaning method for drug-target interaction data, which comprises the following steps:
provide an original collection of drug-target interaction data; and
screen and filter the original drug-target interaction data set according to a predetermined cleaning rule to obtain a drug-target interaction data set to be studied, wherein
the predetermined cleaning rule is based on the data structure of the adjacency matrix of the graph.

In a preferred embodiment of the present invention, the predetermined cleaning rules include:
step 1: construct a general data structure;
step 2: selecting a subset data structure in the general data structure, wherein
   the subset data structure includes the data structure of the original drug-target interaction data set;
step 3: convert the subset data structure of the step 2 into a data structure based on the adjacency matrix of the graph; and
step 4: complete data cleaning based on the data structure of the adjacency matrix of the graph.

In a preferred embodiment of the present invention, the predetermined cleaning rule further comprises:
based on the data structure of the adjacency matrix of the graph, completing the logical relationship or calculating the distance. In a preferred embodiment of the present invention, in the data structure, the storage mode of the adjacency matrix of the graph adopts a sparse matrix recorded by row. In a preferred embodiment of the present invention, the data structure of the adjacency matrix of the graph is such that multiple relationships are stored in the adjacency matrix of the same graph.

In a preferred embodiment of the present invention, in the data structure of the adjacency matrix of the graph, use the way of the flag values converted from binary to integer values to store, so that store a plurality of relationships in the same the adjacency matrix of the graph. In a preferred embodiment of the present invention, the obtained drug-target interaction data set to be studied is used for model training. In a specific embodiment of the present invention, the resulting set of drug-target interaction data to be studied can improve the accuracy of the model. In a specific embodiment, the data processing device processes the obtained drug-target interaction data set to be studied for prediction of drug-target interaction.

The second aspect of the present invention provides a cleaning device for drug-target interaction data, which comprises:
a data providing unit configured to provide an original drug-target interaction data set;
a data cleaning unit configured to screen and filter the original drug-target interaction data set according to a predetermined cleaning rule to obtain a drug-target interaction data set to be studied, wherein
   the predetermined cleaning rule is based on the data structure of the adjacency matrix of the graph.

In a preferred embodiment of the present invention, the predetermined cleaning rules include:
step 1: construct a general data structure;
step 2: selecting a subset data structure in the general data structure, where the subset data structure includes the data structure of the original drug-target interaction data set;
step 3: convert the subset data structure of the step 2 into a data structure based on the adjacency matrix of the graph; and
step 4: complete data cleaning based on the data structure of the adjacency matrix of the graph.

In a preferred embodiment of the present invention, in the data cleaning unit, the predetermined cleaning rule further comprises:
based on the data structure of the adjacency matrix of the graph, completing the logical relationship or calculating the distance.

A third aspect of the present invention provides an electronic device including:
a memory and a processor; wherein
the memory is used for storing one or more computer instructions, and
when the one or more computer instructions are executed by the processor, the method for cleaning the drug-target interaction data according to any one of the present invention is realized.

The present invention can bring at least one of the following beneficial effects:
The present invention provides a scheme for properly describing and comparing the structure, function, etc. of drug target proteins. Under this scheme, differences in target proteins can be quantified. Based on this quantified difference, proper classification of targets can be achieved, or the exclusion index of data sets can be formulated, the data sets can be standardized, eliminate contradictory information, improve model accuracy, and improve model accuracy. The purpose of reducing computational overhead, etc., can help researchers better understand the mechanism behind the data and bring clear information to research. And get better performance in model training.

### BRIEF DESCRIPTION OF DRAWINGS

The preferred embodiments will be described below in a clear and easy-to-understand manner with reference to the drawings, and the above-mentioned characteristics, technical features, advantages and implementations thereof will be further described.

FIG. 1 shows the schematic diagram of the core algorithm principle of the data cleaning method of the present invention; and

FIG. 2 shows a specific embodiment of the data cleaning method of the present invention, and shows a schematic diagram of the data structure of the target protein constructed by a standard terminology system.

### DETAILED DESCRIPTION

Various aspects of the invention are described in further detail below.

Unless otherwise defined or described all professional and scientific terms used herein have the same meanings as those familiar to persons skilled in the art. In addition, any method and material similar or equivalent to those described can be used in the methods of the present invention.

Terminology is explained below.

Unless otherwise clearly specified and limited, “or” described in the present invention includes the relationship of “and”. The “and” is equivalent to the Boolean logical operator “AND”, the “or” is equivalent to the Boolean logical operator “OR”, and “AND” is a subset of “OR”.

It will be understood that, although the terms “first,” “second,” etc. may be used herein to describe different elements, but these elements should not be limited by these terms. These terms are only used to distinguish one element from another. Therefore, a first element could be called a second element without departing from the teachings of the present inventive concept.

In the present invention, the term “containing”, “including”, or “comprising” means that various ingredients may be employed together in the mixture or composition of the present invention. Therefore, the terms “consisting essentially of” and “consisting of” are included in the term “containing”, “including”, or “comprising”.

Unless otherwise clearly specified and limited, terms “link”, “connect”, and “connection” should be understood in a broad sense. For example, the terms may be used for a fixed connection, a connection through intermediate media, an internal connection between two elements, or an interaction relationship between two elements. Persons of ordinary skill in the art may understand specific meanings of the terms in the embodiments of this application based on specific cases.

For example, if an element (or component) is called to be on, coupled with or connected to another element, then the one element may be directly formed on, coupled with or connected to the other element, or there may be one or more intervening elements between them. On the contrary, if the expressions “directly on . . . ”, “directly coupled with . . . ” and “directly connected with . . . ” are used here, it means that there are no intervening elements. Other words used to explain the relationship between elements should be similarly interpreted, such as “. . . ” and “directly between . . . ”, “attached” and “directly attached”, “adjacent” and “directly adjacent”, and so on.

In addition, the words “front”, “back”, “left”, “right”, “up” and “down” used in the following description refer to directions in the drawings. The terms “inside” and “outside” used in refer to the direction towards or away from the geometric center of a specific part, respectively. It is understood that these terms are used here to describe the relationship of one element, layer or region with respect to another element, layer or region as shown in the drawings. In addition to the orientations described in the drawings, these terms should also include other orientations of devices. Other aspects of the invention will be obvious to persons skilled in the art due to the disclosure herein.

In order to explain the embodiments of the present invention or the technical solutions more clearly in the prior art, the embodiments of the present invention will be described below with reference to the drawings. Obviously, the drawings in the following description are only some embodiments of the present invention. For persons of ordinary skill in the art, other drawings and other embodiments can be obtained according to these drawings without any creative effort.

It should also be noted that the illustrations provided in the following examples illustrate the basic concepts of the present application by way of illustration only. The drawings only show the components related to the present application and are not drawn according to the number, shape and size of the components in the actual implementation. The type, number and proportion of each component in the actual implementation may be changed at will, and the layout type of the components may be more complex. For example, the thickness of elements in the drawings may be exaggerated for clarity.

Example: in the present prior art, the premise of cleaning such data is often to classify target proteins, and then select and remove certain categories. There are many existing schemes to describe or classify the structure and function of targets, but there are generally two directions. The two common scenarios for cleaning drug-target interaction data, and the processing schemes of corresponding solutions adopted to solve the problem are shown below:

Scenario 1. Clean the data of the target protein according to the customary classification method

Habit classification method, that is, labeling a group of proteins according to habits, such as classifying and labeling them as: receptors, enzymes, kinases, DNA binding proteins, cytokines, etc. The method is simple, direct and easy to understand.

However, due to the high complexity of biological systems, there are two problems with such a simple division: First, such classification labels are logically unclear. For example, receptor tyrosine kinase proteins commonly used as targets, are both receptors, which is also a kinase, no matter whether this type of protein is labeled alone or mixed with multiple labels, it cannot well reflect its difference and connection with other proteins; second, such labels are often too rough, and a certain type of protein may follow a certain type of protein. It is a common principle, but it is completely different in the specific details of the role, and the habit classification method cannot solve these problems very well.

Scenario 2. Clean the target protein data according to a relatively standardized terminology system

In order to solve the problem of habit classification method, researchers often try to establish a set of relatively standardized terminology system to achieve a more reasonable description of proteins, etc. Such as GO (gene ontology), protein family, MeSH Heading, etc. This terminology system can systematically describe the structure, function, indications and other information of protein targets.

However, such a system often has many entries, and the relationship is very complex. For example, there are about 50,000 entries in the GO system, and each entry may contain another entry in whole or in part; the description of a function may affect, or be specific to up-regulation or down-regulate another function; the same protein may have multiple functions, and these entries may all be derived from an upper function, two proteins may have two different functions, but the two functions may be close (from the same synonym) or decidedly different (from a different branch). There are serious obstacles to interpreting these terms without a strong professional background and a systematic approach. Even in authoritative databases, the entries involved in a certain target and the logical descriptions behind these entries are often not systematic or lacking. If necessary, it may be necessary to refer to the results given by integrating multiple databases and tools, but this must solve the problem of inconsistency between databases.

Another problem of scenario 2 is the difficulty of quantitative analysis. Some tools, although can use the structure of trees and graphs to help visualize the complex relationships between entries, they are often only suitable for browsing, and cannot give quantitative evaluations of targets. Usually, may process a series of entries into vector elements with a value of 0/1, and then perform distance calculation or classification accordingly. However, this method cannot reflect the complex logical relationship between entries.

In order to solve the problems existing in the above scenarios, in the present invention, the inventors have undergone extensive and in-depth experiments to provide a scheme for properly describing and comparing the structures and functions of drug target proteins. Under this scheme, the differences in target proteins can be quantified. Based on this quantified difference, can achieve the proper classification of targets, or formulate exclusion indicators of datasets, realize datasets standardization, eliminate contradictory information, improve model accuracy, and reduce the computational overhead and other purposes, which can help researchers better understand the mechanism behind the data and bring clear information to research. And get better performance in model training.

The present invention further describes the technical path of the concrete realization of the establishment of the data structure as follows. It should be understood that all the following descriptions are exemplary, and various aspects of various technical solutions can be combined to obtain other optional technical solutions. Various aspects of the present invention are described in detail below with reference to the accompanying drawings.

The first aspect of the present invention provides a cleaning method for drug-target interaction data, which comprises the following steps:
Provide an original collection of drug-target interaction data; and
Screen and filter the original drug-target interaction data set according to a predetermined cleaning rule to obtain a drug-target interaction data set to be studied, wherein
   the predetermined cleaning rule is based on the data structure of the adjacency matrix of the graph.

The inventors found that by establishing a graph-based adjacency matrix data structure, the differences of target proteins can be quantified, so as to provide a premise for the present invention to establish a reasonable data structure for the present invention, clarify complex logical relationships, and give full play to the due value of these standard terminology systems.

Further, the inventors found that, on the premise that the differences in the target proteins can be quantified, based on this quantified difference, can achieve the proper classification of targets, or formulate exclusion indicators of datasets, realize datasets standardization, eliminate contradictory information, improve model accuracy, and reduce the computational overhead and other purposes, that can help researchers better understand the mechanism behind the data and bring clear information to research. And get better performance in model training.

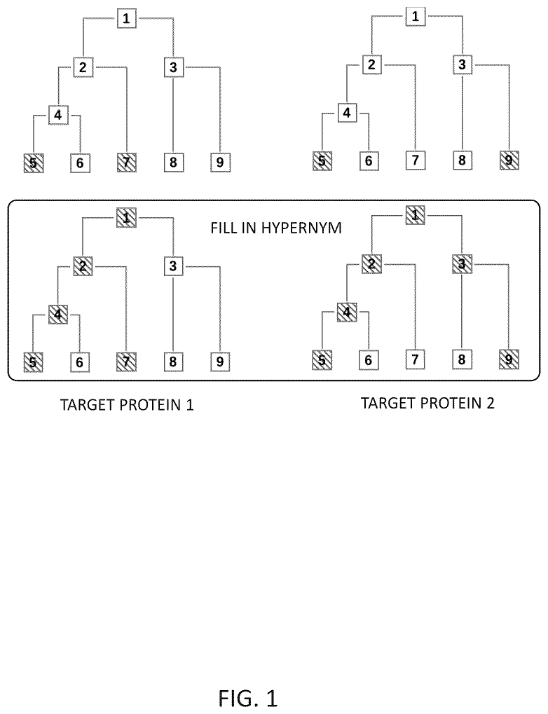

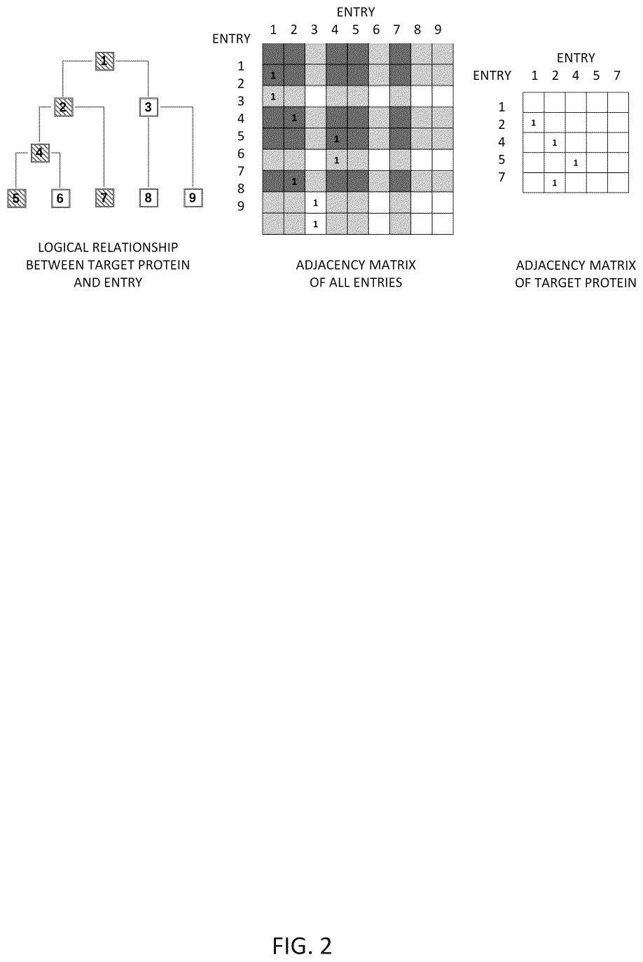

Referring specifically to FIG. 1, it shows a schematic diagram of the core algorithm principle of the data cleaning method of the present invention; FIG. 2 shows a specific embodiment of the data cleaning method of the present invention, showing that the data of the target protein is constructed by a standard terminology system. Schematic.

In a specific embodiment of the present invention, the predetermined cleaning rules include:
Step 1: construct a general data structure;
Step 2: selecting a subset data structure in the general data structure, where the subset data structure includes the data structure of the original drug-target interaction data set;
Step 3: convert the subset data structure of the step 2 into a data structure based on the adjacency matrix of the graph; and
Step 4: complete data cleaning based on the data structure of the adjacency matrix of the graph, wherein
   in step 1, a general data structure (a data structure for constructing entries relationships) is constructed.

Exemplarily, first construct a general (abstract) data structure format class that stores the entry system. It should be understood that the present invention does not have mandatory requirements on the specific implementation method of the data structure, and various computer languages can easily construct it, or a similar data structure can be used as its base class. In a preferred embodiment of the present invention, in the data structure, the storage mode of the adjacency matrix of the graph adopts a sparse matrix recorded by row.

The function of the sparse matrix is: because the number of entries is usually large, but the number of logically associated entries for each entry is limited, for memory usage and efficiency, the present invention uses a sparse matrix recorded by row as the storage mode of the adjacency matrix of the entry relationship. Specifically, because the number of entries is usually large, but the number of logically related entries of each entry is limited, for the sake of memory usage and efficiency, the invention uses the sparse matrix recorded by rows as the storage mode of the adjacency matrix of entry relationship. Because for the application of the invention, the search logic relationship is asymmetric, generally speaking, the present invention needs to find the hypernym of the entry instead of the lower sense word, and the sparse matrix recorded by rows can greatly reduce the number of cycles, which will be reduced to about 20,000 for a conventional application, thus greatly improving the computational efficiency. (If necessary, if a large number of semantic words need to be searched, the data structure can also be converted into a sparse matrix recorded by columns, or other common sparse matrix forms. There are ready-made algorithm implementations for reference.)

It should be understood that the storage mode of the adjacency matrix of the graph in the form of recorded by rows is the optimal choice, and the advantages include that there are ready-made methods to convert sparse matrices of different forms. However, the storage mode of the adjacency matrix of the graph can also be converted to other forms when necessary.

For example, when specific operations are required, other suitable sparse matrix forms can be used for storage. In a specific embodiment of the present invention, based on the principle of symmetry, the form of a sparse matrix recorded by columns is also feasible.

Exemplarily, this data structure is inherited from the “sparse.lil” matrix class of the “scipy” module of python, mainly because the scipy module has implemented common computations for sparse matrices, which can reduce the workload of method rewriting. But theoretically a similar data structure in any language can be used as the base class for this data structure.

Specifically, the matrix elements of the sparse matrix are used to store the relationship between the entries. Usually, the value of the j-th element in the i-th row is not 0, which means that the entry j is the hypernym entry under a certain logical relationship of the entry i. In a preferred embodiment of the present invention, the data structure of the adjacency matrix of the graph is such that multiple relationships are stored in the adjacency matrix of the same graph. In a specific embodiment of the present invention, in order to store a plurality of relationships in the same matrix, the flag values converted from binary to integer values are stored.

For example, if a data set has four logical relationships, the relationship can be represented by a four-bit binary number, and each relationship will correspond to one digit of the binary. Relation 1 corresponds to the zeroth position of 2, relation 2 corresponds to the first position of 2, and so on. When the relationship does not exist, the digit is 0, and when it exists, it is 1. Therefore, when the first and fourth relationships exist between the two entries, the value is Ob1001, and the corresponding integer value is 9. This class also contains at least two instance data: one is a hash-based index (such as python's ordered dictionary), which is used to store each entry and its corresponding serial number; the second is a relational comparison table, which is used to store the binary bit value corresponding to each relationship.

A dataset that specifically stores a certain terminology system may also require other instance data. For example, GO entries are divided into three different categories. Each category has a root main entry, and each entry also has a textual description. The class go format needs some additional content, which can be added on the basis of inheriting the above data structure.

As mentioned above, the present invention has no mandatory requirements for the specific implementation method of the data structure, and various computer languages can construct it conveniently, or similar data structures can be used as its base class. As long as there is no restriction on the inventive purpose of the present invention. More specifically, the data structure needs at least the following methods to meet the analysis requirements:

Construction function, which reads the hash table of the relation between the entry list and the entry, and initializes the index, adjacency matrix and relation comparison table. It can also be constructed by a sparse matrix. Rewritten the addition and multiplication operations, in python, which are four functions, _add_, _radd_, _mul_ and _rmul_, so that two instances with different logical relationships can be merged, and the results still return the correct data type. The typical way is to still use the arithmetic function of the sparse matrix itself for operation, and call the constructor again to generate the return value with the correct type. The rewritten indexing function, in python, is _getitem_ and _setitem_, so that it can be indexed by entries instead of only by the row and column numbers of the matrix.

The functions get_parents and get_children that read the hypernym and the hyponym of a certain entry, given the entry, and one or more logical relationships, get all parents entries or children entries that match the relationship, specifically, according to the numerical digit corresponding to the logic relationships, perform the bitwise and operation to the corresponding matrix element, and the result is not 0, indicating that the relationship exists. From this, obtain all the related elements (corresponding entries) of the rows or columns of the matrix.

Read the function get_all_parents of all the hypernyms of an entry, given the entry and logical relationship, read upwards circularly until the entry has no hypernym (root main entry), record and return the found entry list. In order to be efficient and avoid the number of layers exceeding the limit, the loop must adopt a non-recursive mechanism, that is, the traversal is realized by pushing and extruding the heap.

In addition, some auxiliary out-of-class functions are established to help data construction, which is caused by the different data file forms of different terminology systems. But the core idea is to read all the entries, all the logical relationships, and what logical relationship each entry is connected with. Specifically, you can first obtain the relationship between all the entries and the entries of a terminology system used for protein structure or function.

Take GO as an example:

The download page of official website (http://geneontology.org/docs/download-ontology/) can get the data set of entries, and for some data in the data set, it also records the hypernym entries of each entry in a certain relationship. Read these records step by step circularly. In step 2, select a subset data structure in the general data structure, and the subset data structure includes the data structure of the original drug-target interaction data set. Specifically, step 2 may create examples that record the entries involved in a particular protein. The data structure of step 2 is a subset of the data described in step 1, that is, the partial entries (involving the target protein to be studied) and their adjacency matrices are stored.

Specifically, the overall shape and basic functions of the data structure are the same as the above, and can establish the member function get_sub_mat to create. More specifically, the function takes as input the sequence of entries involved. For these entries, the constructor of the format class is called to form an instance of an empty sparse matrix, and then extract the involved rows and columns to fill in the missing values of the adjacency matrix.

Step 3 converts the subset data structure of step 2 into a data structure based on the adjacency matrix of the graph (i.e., performs quantitative calculation).

In the present invention, a series of quantitative analysis methods can be used. The quantitative methods may be employed simultaneously or separately to obtain the graph-based adjacency matrix data structure. Specifically including:

The edit distance is used as a measure of the protein difference between the two targets. Since all the hypernyms of a specific target have been supplemented in the data structure described in step 2, the edit distance is the sum of the difference between the instance entries corresponding to the two targets.

Or, screen for close (or dissimilar) targets: given an instance of a target and a threshold of distance, any distance within (or outside) the threshold can be selected.

Or, perform a cluster analysis. Since the present invention defines a calculating method of the distance between two target points, apply a density-based clustering algorithm to perform a clustering operation on them. First, iterate from any instance, all the distances within the set threshold range can be included in the same class, and iterate until all instances have been processed. In a specific embodiment of the present invention, in addition to the above-mentioned core computing functions, the present invention also introduces additional computing functions.

Specifically, obtain all the connection paths between certain two entries, obtain the number of paths and the length of each path, the function first obtains all the paths of each entry leading to the main entry, and finds out the path which involves another entry, and then record the part between the two entries, and then perform the same operation on the other entry, merging and removing duplicates. More specifically, the process of obtaining the path can be recursive or non-recursive.

Preferably, in view of the possibility of a large number of entries and the consideration of efficiency, the present invention can use a non-recursive manner, that is, each entry is processed, it is pushed into the heap, and remove it after at the end of processing, and the circulate until the heap is empty. The inventor found that by converting the data into a graph, if necessary, the structure of the graph can better reflect the relationship between the entries than the adjacency matrix. More specifically, the present invention can construct an indexable graph data structure as an aid.

For example, the data structure can be implemented by inheriting python's abstract containers, such as sequence, etc. (or other abstract containers in other languages). The function traverses all involved entries and reads their hypernym and the hyponym entries, then updates the structure of the graph. In order to facilitate the analysis of the data, other contents are also recorded. In a specific embodiment of the present invention, starting from the root main entry (or the most hyponym entry), the level where the entry is located can be updated step by step and recorded. Such as the level of each entry in the graph, it is convenient to query more basic and general entries, or more specific and more outstanding entries.

Step 4: complete data cleaning based on the data structure of the adjacency matrix of the graph.

In a preferred embodiment of the present invention, the obtained drug-target interaction data set to be studied is used for model training. In a specific embodiment of the present invention, the resulting set of drug-target interaction data to be studied can improve the accuracy of the model. In a specific embodiment, the data processing device processes the obtained drug-target interaction data set to be studied for prediction of drug-target interaction. Specifically, for example, process protein classification and complete the data screening based on the data structure of the adjacency matrix of the graph. Specifically, the authority record identifier of the protein should be obtained first. Exemplarily, use the protein ids from the Uniprot database.

Specifically, the present invention produces a module for fuzzy search based on name, and a module for query based on gene name, and a module for corresponding gene aliases to standard gene names (symbol corresponding to Entrez gene id), which can meet most routine applications. Since this part only requires an identifier to be output, if there are special requirements, you can also easily write a query tool or use existing resources to query, and then integrate into the existing system.

As mentioned above, the present invention obtains a list of uniprot ID, and for each element, queries the corresponding entry under its corresponding classification system. The method for obtaining the uniprot data set (which can be downloaded and updated in official website) is compiled in this invention.

It should be noted that which entries in a protein conform to the terminology system are updated with the research progress, and are maintained by different organizations. As for the specific GO entry, the record of uniprot is not consistent with the official record of GO. Technically, the present invention can obtain these entries for a target protein in many ways, but here uniprot is mainly used for consistency. Because this part only needs to output a list of entries, if you have special requirements, you can also easily write query tools or use existing resources to query, and then integrate them into the existing system.

Then, for each target, the invention constructs an example of the data structure described in step 2 through the example constructed in step 1 and the above-mentioned obtained entry list. Then, for each target, calculate its distance in pairs.

If the model is to predict the drug structure-activity relationship of a protein, firstly, the data set of the target close to it is screened out, and the data set far away from it is removed, and then the conventional machine learning modeling is carried out.

Compared with the conventional data cleaning method, the invention is different in that:
1. Establish a data structure, which can include all the entries and logical relationships in a certain terminology system, and provide necessary operation methods, including finding the upper and lower semantic entries according to the logical relationship, refining the logical path between some two entries, and performing operations on different instances of the data structure, such as subtraction, merging, etc.
2. For a certain protein target, obtain all the entries involved in it in an authoritative way at present, and then instantiate the above data structure for the protein to fill in the logic deficiency. For two proteins, we can describe the differences between two examples, compare and classify them, remove interference or select similar targets.

In addition, as a method framework, the invention realizes the decoupling of operation and data, so it can be applied to various data sets with similar structures, and it can also be conveniently updated synchronously when the data sets change.

More specifically, the present invention cites the following data cleaning methods.

Here, the data cleaning method of the present invention is shown by taking the prediction of the drug effect of CDK1 (Uniprot ID: P06493) (predicting Y by X) as an example. The initial data set of this embodiment is as follows:

Select 72 widely studied proteins (including the protein to be predicted), and their uniprot IDs are:
P00519,P22303,P78536,P31749,P27338,014965,P56817,O60885,P15056,Q06187,P4257 4,P07339,P51681,P06493,P24941,O14757,P06276,P27487,P00533,P08246,P04626,Q92731,P03 372, P00742,P11362,P49354,P49841,Q13547,Q92769,P34913,P08069,P05556,P05106,P23458, O 60674,P52333,O60341,P43405,Q07820,Q00987,P08581,P28482,Q16539,P03956,P45452,P1478 0,P43490, P04629,P41145,043614,043613,P27986,P09874,Q07343,Q08499,P27815,076074,P1 1309,000329,P48736,P42336,O14684,P18031,P04049,P00797,075116,P51449,P31645,Q15858, P12931,P00734, P35968.

The ic50 test results of 1000 drugs or potential drug molecules are randomly selected from the chembl database for each protein (if there are multiple results, 10% will be truncated at both ends and the average value will be taken). The -log value of 8 is used as the threshold, which is positive when it is higher, and negative when it is lower. Use this as the y value

The protein is converted into the SMILES form according to the amino acids in the ligand-binding region marked in its PDB data, and the drug or potential drug is subject to the SMILES form provided by the database. Construct the fingerprint by using the standard method of the software rdkit, and use this as the X value.

The cleaning steps are as follows:
1. The terminology system used in this case is GO, and its data is downloaded from the official website. Each record in this data set includes the GO entry identification, description, alias, and logical relationship, whether it is deprecated or not. Here, the present invention only collects entries that are not deprecated, and all aliases are converted to classic names. The reading scheme is relatively straightforward, with the identification as the key and the rest of the information as the value. The logical relationship part records what kind of logic, and the identifier of the related entry. Logical relationship in this embodiment only takes two parts: is a and is part of. Pass that dictionary to the constructor of the format class to instantiate it.
2. For each uniport id, in the uniprot data set (downloaded from the official website), get the GO entries involved. If there is an alias entry, convert it to the classic entry name, and remove the discarded entries.

3. For a set of GO entries corresponding to each uniprot id, call the get_all_parents method of the instance described in step 1, supplement the complete logical relationship, and call the get_sub_mat method of the instance to create a sub-instance for the uniprot id.

4. For the protein to be predicted, calculate the distance between the instance corresponding to its uniprot id and the other 71 instances, and select the nearest 12 and the farthest 12.

5. Extract the nearest 12 proteins (group 1), all 71 proteins (group 2), and the farthest 12 proteins (group 3) from the original data set as training data.

### Model Training and Results:

Use the random forest method, set min_samples_leaf=2, n_estimators=1000, train the

protein-drug data of each group, and use the X data of the protein to be studied for prediction, compare with its real y data, and calculate the AUC value of ROC curve.

As a result, the AUC values of group 1 are 0.874, group 2 are 0.854, and group 3 are 0.674

It can be seen that selecting the closest protein yields the best results, slightly better than using all proteins, and significantly better than selecting proteins with greater differences. At the same time, due to the small training data set, in terms of model training speed, group 1 has about 5 times improvement compared to group 2. The above results prove that the data cleaning method of the present invention can achieve both effect and efficiency.

To sum up, the specific embodiments of the present invention have obtained the following effects:
1-This technology can effectively utilize the complex standard terminology system to achieve functional interpretation of protein targets, as well as applications such as classification and screening.
2-Compared with the traditional vector-based quantitative analysis method of entry system, this technology retains the logical relationship of entries and utilizes information more comprehensively.
3-Data screening through this technology can more effectively clean the data set, eliminate contradictory interfering data, and improve the performance of the model.

The second aspect of the present invention provides a cleaning device for drug-target interaction data, which comprises:
A data providing unit configured to provide an original drug-target interaction data set; and
A data cleaning unit configured to screen and filter the original drug-target interaction data set according to a predetermined cleaning rule to obtain a drug-target interaction data set to be studied, wherein
   the predetermined cleaning rule is based on the data structure of the adjacency matrix of the graph.

In a preferred embodiment of the present invention, in the data cleaning unit, the predetermined cleaning rule further comprises, based on the data structure of the adjacency matrix of the graph, completing the logical relationship or calculating the distance.

A third aspect of the present invention provides an electronic device including a memory and a processor; Wherein the memory is used for storing one or more computer instructions, and when the one or more computer instructions are executed by the processor, the method for cleaning the drug-target interaction data according to any one of the present invention is realized.

Based on the present application, persons skilled in the art should understand that an aspect described herein can be implemented independently from any other aspects, and two or more of these aspects can be combined in various ways. For example, any number of aspects set forth herein can be used to implement devices and/or to practice methods. In addition, other structures and/or functionalities other than one or more of the aspects set forth herein may be used to implement the device and/or to practice the method.

Persons skilled in the art shall understand that, in addition to implementing the system provided by the present invention and its various devices, modules, and units in a pure computer-readable program code manner, they may also be implemented to realize the same functions in the form of logic gates, switches, application-specific integrated circuits, programmable logic controllers, embedded microcontrollers, and the like, by performing logic programming on the steps of the method. Therefore, the system provided by the present invention and its various devices, modules and units can be regarded as hardware components, and the devices, modules, and units included in the system for implementing various functions can also be regarded as structures within the hardware components. Besides, the devices, modules, and units for realizing various functions can also be regarded as not only software modules but also the structures within the hardware components for implementing the method.

It should be noted that the above examples can be freely combined as required. The above are merely preferred embodiments of the present invention. It should be pointed out that for those of ordinary skill in the art, several improvements and modifications can be made without departing from the principle of the present invention, and these improvements and modifications should be regarded as the protection scope of the present invention.

All documents mentioned in the present invention are incorporated herein by reference, as if each one is individually incorporated by reference. Additionally, it should be understood that after reading the above teachings, persons skilled in the art can make various changes and modifications to the present invention. These equivalents also fall within the scope defined by the appended claims.

1. A cleaning method for drug-target interaction data, characterized in comprising the following steps:
provide an original collection of drug-target interaction data; and
screen and filter the original drug-target interaction data set according to a predetermined cleaning rule to obtain a drug-target interaction data set to be studied, wherein
the predetermined cleaning rule is based on characteristics of a data structure of an adjacency matrix of a graph.

2. The cleaning method for drug-target interaction data according to claim 1, wherein the predetermined cleaning rule includes:
Step 1: construct a general data structure;
Step 2: selecting a subset data structure in a general data structure, where the subset data structure includes the data structure of the original drug-target interaction data set;
Step 3: convert the subset data structure of step 2 into a second data structure based on the adjacency matrix of the graph; and
Step 4: complete data cleaning based on the data structure of the adjacency matrix of the graph.

3. The cleaning method for drug-target interaction data according to claim 1, wherein
based on the data structure of the adjacency matrix of the graph, executing at least one element of a set comprising completing a logical relationship and calculating a distance.

4. The cleaning method for drug-target interaction data according to claim 1, wherein
a storage mode of the adjacency matrix of the graph adopts a sparse matrix recorded by row.

5. The cleaning method for drug-target interaction data according to claim 1, wherein
the data structure of the adjacency matrix of the graph is such that multiple relationships are stored in the adjacency matrix of the same graph.

6. The method for cleaning drug-target interaction data according to claim 5, wherein
in the data structure of the adjacency matrix of the graph, use the way of the flag values converted from binary to integer values to store, in order to store a plurality of relationships in the same as the adjacency matrix of the graph.

7. A cleaning device for drug-target interaction data, characterized in comprising:
a data providing unit configured to provide an original drug-target interaction data set; and
a data cleaning unit configured to screen and filter the original drug-target interaction data set according to a predetermined cleaning rule to obtain a drug-target interaction data set to be studied, wherein
the predetermined cleaning rule is based on the data structure of the adjacency matrix of the graph.

8. The cleaning device for drug-target interaction data according to claim 7, wherein the predetermined cleaning rules include:
Step 1: construct a general data structure;
Step 2: selecting a subset data structure in the general data structure, wherein the subset data structure includes the data structure of the original drug-target interaction data set;
Step 3: convert the subset data structure of step 2 into a data structure based on the adjacency matrix of the graph; and
Step 4: complete data cleaning based on the data structure of the adjacency matrix of the graph.

9. The cleaning device for drug-target interaction data according to claim 7, wherein
in the data cleaning unit, the predetermined cleaning rule further comprises executing at least one element of a set comprising, based on the data structure of the adjacency matrix of the graph, completing the logical relationship and calculating the distance.

10. An electronic device, characterized in including:
a memory and a processor, wherein
the memory is used for storing one or more computer instructions, and wherein when the one or more computer instructions are executed by the processor, the method for cleaning drug-target interaction data characterized in comprising the following steps:
provide an original collection of drug-target interaction data; and
screen and filter the original drug-target interaction data set according to a predetermined cleaning rule to obtain a drug-target interaction data set to be studied, wherein
the predetermined cleaning rule is based on characteristics of a data structure of an adjacency matrix of a graph.
